(19) United States
(12) Patent Application Publication
(10) Pub. No.: US 2008/0248936 A1
(43) Pub. Date: Oct. 9, 2008

(54) Therapeutic Neutral Spine and Exercise Device and Method of Applying Same

(75) Inventors: Nadine FERRISS, Ottawa (CA); Tammy Boucher, Ottawa (CA)

Correspondence Address: NELLIGAN O'BRIEN PAYNE LLP, 66 SLATER, SUITE 1900, OTTAWA, ON, K1P-5H1, CA

(73) Assignee: 6545998 CANADA INC., Ottawa (CA)

(21) Appl. No.: 11/696,227

(22) Filed: Apr. 4, 2007

(51) Int. Cl. A63B 26/00; A61F 5/00

(52) U.S. Cl. 482/142; 602/19

(57) ABSTRACT

An “I” shaped therapeutic neutral spine and exercise device for improving muscle balance and soft-tissue and dural/nerve mobility by encouraging and facilitating proper neutral position of the entire spine is disclosed. The device has three sections of different dimensions; a cervical, thoracic and lumbar section. Each section is placed at a specific area of the spine. Each section can be utilized independently or as a whole by health care providers while administering manual therapy techniques or by users themselves by self-administering stretches. Each section is filled with grain, bean, pea, shell or synthetic substance with optionally attachable tabs to allow the user to utilize each section independently or the device as a whole. The device comes in various sizes to accommodate various lengths of the spine. Methods of administering the therapeutic neutral spine and exercise device on users by health care professionals for enhancing neutral spinal position, improving muscle balance, soft tissue and nerve tissue tension are also disclosed.

### FIELD OF THE INVENTION

The present invention is directed to a therapeutic neutral spine and exercise device which improves muscle balance and soft-tissue and dural/nerve mobility of a user through exercising and facilitating proper spinal alignment. The present invention also assists health care providers to administer manual therapy techniques by employing the therapeutic neutral spine and exercise device.

### BACKGROUND OF THE INVENTION

There are many dysfunctions that can develop secondary to muscle imbalances, soft-tissue tightness and dural/nerve tissue tightness. TMJ dysfunction, nerve entrapments, thoracic outlet syndrome, headaches, facet syndromes and the appearance of trigger points are to name a few. Muscle imbalances can be the cause of any number of muscle, fascia, metabolic and mechanical dysfunctions and syndromes. Left untreated, these imbalances can lead to degenerative joint conditions secondary to the excessive forces exerted on the joints, ligaments and discs from overactive and opposing underactive muscles.

The muscles that surround human joints are coupled with opposing muscles of equal force. These forces have a certain direction and strength so a change or imbalance in one of these forces will alter joint position and function and eventually lead to poor joint mechanics, joint restriction, improper musculoskeletal movement and joint breakdown. The poor joint mechanics will eventually lead to joint and soft tissue irritation, pain and stiffness.

When properly balanced human muscles work together to allow the body parts to move in a painfree and non-restricted way. When muscle imbalance is present, a muscle or group of muscles are hypertonic and the opposing muscles become hypotonic. A muscle that is hypertonic is a muscle that is shortened, tight and in a constant state of contraction. If this hypertonic state persists, it can develop into a muscle that is in a chronic state of contraction and the body eventually recognizes this as a “normal” state. A muscle that is in a chronic state of contraction can squeeze the blood vessels and nerve fibers in the muscle tissue and decrease proper nourishment and nerve conductivity that is required for the muscle to function properly. The opposing hypotonic muscle becomes stretched and weak and has difficulty contracting. This imbalance leads to altered joint mobility and eventually breakdown.

Poor posture, jobs that require static or repetitive movement, lack of exercise, over exercise and incorrect exercising, accident and surgery are common causes of muscle imbalances and dural tightness. If the issue is not addressed properly, the body will adapt to the changes. In order to restore balance, the shortened muscle must be stretched and the lengthened muscle must be strengthened. Devices that have been used to help address this problem is taping and bracing. These techniques have not proven to be effective over long periods of time. They are also inconvenient since they occasionally require the assistance of a second party to apply. Also, some individuals are allergic to the tape adhesive.

Soft-tissue tightness secondary to post-surgical scarring can be problematic as well. Scarring and adhesions are a normal part of the natural healing process but can become problematic post-surgery if adhesions form around the tissue, nerves and organs causing them to bind and tether to the surrounding tissue. Post-surgical scars can become crythematous, raised and painful. This issue must also be addressed with proper stretches.

The dura is the external lining of the spinal canal. It is continuous with the meninges of the brain. The nerve sheath that surrounds and protects the peripheral nerves is also a lining that begins at the dura. This nerve tissue responds and adapts to all biomechanical changes in the human body. Treating nerve tissue tension is a necessity when a dysfunction or syndrome is present. Treatment requires precision and exercises must not be aimed at overstretching the tissue.

Health care providers such as physiotherapists, massage therapists, chiropractors and kinesiologists attempt to address the above-mentioned ailments. But, at this present time an effective means to assist the user in properly addressing these ailments in an independent fashion while maintaining proper spinal alignment is not available. Also, a means to assist health care providers in administering their manual therapy techniques while addressing these ailments is not available.

There are a few U.S. patents and published applications that describe a device or a method for stretching and posture improvement. These devices and methods all attempt to support and improve posture and stretch specific muscle groups but very few of them push the entire spine into neutral position from the cervical to the lumbar spine. Neutral spinal position being defined as proper curvatures of the spine at rest.

For example, the device described in the U.S. Pat. Appln. No. 2005/0131462 addresses only the upper section of the thoracic spine. Incorporation of the lower thoracic spine, cervical and lumbar spine has not been taken into consideration and the device is not used for exercise purposes. In addition, this device has a concave configuration and provides a longitudinal groove to accommodate bony prominences of the vertebrae. The device also pushes the thoracic spine into mild hyperextension. U.S. Pat. No. 6,902,537 describes an upper body support device addressing only the cervical and thoracic regions of the spine and is not described as being used for exercise purposes. Furthermore, neutral position of the cervical and thoracic spine are not taken into consideration.

U.S. Pat. No. 5,099,831 describes an exercise device isolating only the thoracic spine for means of improving posture and stretching. The user must exert a force with their arms elevated which could cause shoulder dysfunctions. Also, with this device, a proper stretch cannot be maintained for a sufficient amount of time to be effective. The human body must be in a relaxed state in order to properly stretch soft-tissue.

Similarly, U.S. Pat. Nos. 5,429,585 and 5,033,137 disclose support devices which specifically support the user's lower back and cervical region, respectively. These support devices cannot be used for exercise purpose at all.

There exists some prior art devices designed to accommodate the whole spine. For instance, U.S. Pat. No. 4,572,578 provides a backrest in the shape of an “S” to conform, support and provide comfort to the cervical, thoracic and lumbo-sacral spine. The thoracic component is concave with two sections to accommodate to the bony prominence. The cervical component is an inverted U-shape to conform to the lordosis of the neck. The lumbar-sacral section is convex to conform to the lordosis of the lower back and is equipped with a kidney support. However, the rigidity of this prior art device prevents the user from performing stretching exercises and is not conducive in pushing the spine to neutral position as it only supports the spine. Such a device is also complicated to manufacture and expensive for retail sales.

U.S. Pat. Appln. No. 2003/0159698 teaches a long tubular gel-filled device, cylindrically shaped from end to end. The device is placed lengthwise along the entire spine to support the existing curvatures. The gel-filled device molds to the existing curvatures and applies an evenly distributed pressure but fails to correct spinal position and facilitate neutral spine. Also, its configuration fails to properly support the soft tissue that surrounds the cervical and lumbar vertebrae. For this reason, an “I” shaped configuration is preferred as such configuration will facilitate neutral spinal position. Moreover, having the cervical and lumbar sections the same width as the thoracic section, supporting the vertebral bodies only, would be very uncomfortable to lie on and would not allow the user to completely relax on the device.

The above mentioned U.S. patents and published patent applications fail to provide a means by which an individual can properly perform stretching exercises to reverse flexed posture assumed throughout the day all while pushing their entire spine in a neutral position. These prior art also fail in providing a means by which to assist health care providers to administer their manual therapy techniques while encouraging user's proper spinal alignment.

Accordingly, there is a long-felt need amongst both the users and health care providers for a therapeutic neutral spine and exercise device that promotes neutral position of the entire spine whereby the user can lie on it to do proper gravity assisted stretches and exercises independently or the health care providers can administer it on the user to carry out different forms of manual therapies.

### SUMMARY OF THE INVENTION

It is an object of the invention to provide a device that can be used by health care professionals to perform manual therapy techniques to help restore proper joint mobility, muscle balance and nerve tissue mobility while facilitating neutral spinal alignment of the users.

It is another object of the invention to provide a device that can be self administered by users for proper stretching of the appropriate muscle groups, post-surgical incisions and nerve tissue to help restore proper muscle balance, soft-tissue and dural/nerve mobility.

According to one aspect of the invention, it provides an “I” shaped therapeutic neutral spine and exercise device comprising a cervical section, a thoracic section and lumbar section, wherein the cervical section of the device being the length of the cervical spine (C1 to C7) of a user and width corresponding to width of the neck of said user and thickness sufficient to facilitate a neutral position of the cervical spine of said user; the thoracic section of the pillow being the length of the thoracic spine (T1 to T12) of said user and width being no larger than the distance between the scapula of said user and thickness sufficient to facilitate neutral spinal position and allow the shoulders of said user to translate posteriorly; and the lumbar section of the pillow being the length of the lumbar spine (L1-L5) of said user and width corresponding to the width of the low back of said user and thickness sufficient to facilitate a neutral position of the lumbar spine of said user; and the three sections are demountably attached to each other by fastening means.

The three sections provide a linear profile that serves to decrease exaggerated thoracic spine kyphosis.

According to another aspect of the invention, it provides the same “I” shaped therapeutic neutral spine and exercise device except the three sections form an integral unit.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a partial perspective side view of the therapeutic neutral spine and exercise device positioned between the user's spine and a rigid surface.

FIG. 2a is a perspective view of the therapeutic neutral spine and exercise device showing the cervical, thoracic and lumbar sections of the device as an integral unit.

FIG. 2b is a perspective view of the therapeutic neutral spine and exercise device showing the cervical, thoracic and lumbar sections of the device optionally detached.

FIG. 3 is a posterior view of the thoracic section of the therapeutic neutral spine and exercise device.

FIG. 4a is a side view of the therapeutic neutral spine and exercise device showing the cervical, thoracic and lumbar sections of the device optionally detached.

FIG. 4b is a side view of a preferred embodiment of the present invention in which all upper surfaces of the corresponding cervical, thoracic and lumbar sections form a linear profile.

FIG. 5 is a perspective side view of the therapeutic neutral spine and exercise device illustrating a user adjusting himself to lie on the device.

FIG. 6 is a perspective side view of the therapeutic neutral spine and exercise device illustrating a user lying down on the device and adjustment of the cervical section.

FIG. 7 is a perspective side view of the therapeutic neutral spine and exercise device illustrating a user doing a dural/nerve stretch with the device.

FIG. 8 is a perspective side view of the therapeutic neutral spine and exercise device illustrating a user performing a pectoralis stretch with the device.

FIG. 9 is a perspective side view of the therapeutic neutral spine and exercise device illustrating a user performing a latissimus dorsi stretch with the device.

### DETAILED DESCRIPTION OF THE PREFERRED EMBODIMENTS

The present invention is a therapeutic neutral spine and exercise device to promote neutral position of the entire spine. A user can lie on it to do stretches and exercises or a health care provider can administer it on the user to carry out different forms of manual therapies.

In general terms, the therapeutic neutral spine and exercise device of the present invention is a pillow in the shape of a horizontal “I” with a linear configuration to provide proper support to the soft tissue that surrounds the cervical and lumbar vertebrae. An “I” shaped configuration will facilitate neutral spinal position. It is important that the widths of the cervical and lumbar sections corresponds to the width of the neck and lower back accordingly and be greater than the width of the thoracic section, giving it the “I” shape. This particular width will allow the soft tissue (muscles, fascia, ligaments . . . ), along with the vertebrae, to be pushed into neutral spinal position so that they can adopt their normal soft tissue length. The “I” shape also provides stability to the users when they are lying down on it. The wider width at the cervical and lumbar sections stabilizes the body, so that the users do not feel that they are going to roll off the device. This stability allows the users to relax and the device to promote normal soft tissue length. When the lumbar and cervical spine lose their normal lordotic curvature, the soft tissue becomes lengthened and weak. Their proper resting length is encouraged by facilitating neutral spinal position.

The device has three sections, namely the cervical section, the thoracic section and the lumbar section. All three sections are formed as one integral unit or, optionally, as demountable separate sections. When administering the therapeutic neutral spine and exercise device, the goal is to push the user's spine into a neutral position, as opposed to supporting the existing spinal curvature. Since repetitive posture during the day promotes an increased thoracic kyphosis, forward head posture and decreased lumbar lordosis, the pressure applied by the device is not even, and the user may feel a greater amount of pressure along the thoracic spine as he feels his shoulder fall back with the help of gravity, causing a passive stretch to the muscles and nerve tissue.

The therapeutic neutral spine and exercise device of the present invention is also an exercise device which can be used to promote neutral position of the entire spine. The user lays on it to do stretches and exercises. The thoracic component is in a linear shape without any groove in the middle in order to push the bony prominences forward to promote neutral position of the thoracic spine. The cervical component is also in line with the thoracic component and it stops at the occiput to allow the cranium to hang over in order to stretch the sub-occipital ligament. Likewise, the lumbar section is linear in configuration to ensure neutral position of the lumbar spine.

In a preferred embodiment, the sections of the therapeutic neutral spine and exercise device can be optionally detached from one another to facilitate treatment of the specific area(s) of the therapist's choice.

In another preferred embodiment, all upper surfaces of the corresponding cervical, thoracic and lumbar sections form a linear profile.

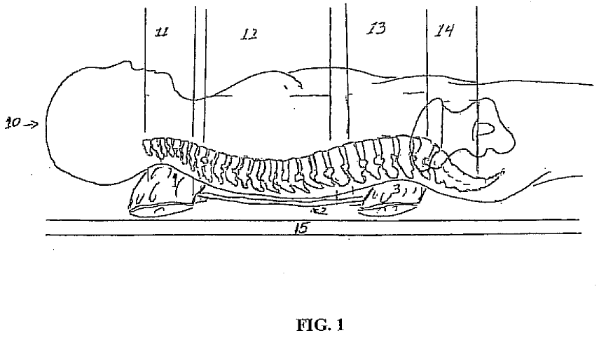

With reference now to the drawings, FIG. 1 shows the device with three sections: cervical section 1, thoracic section 2 and lumbar section 3, positioned between the user's spine (cervical spine 11, thoracic spine 12 and lumbar spine 13) and a rigid surface 15. Each section of the therapeutic neutral spine and exercise device is placed at a specific area of the spine corresponding to that section. As shown in FIG. 1, a user 10 is depicted thereon. FIG. 1 also identifies the user's cervical spine 11, thoracic spine 12, lumbar spine 13 and sacrum and coccyx 14.

The exterior of the sections 1, 2 and 3 are made of conventional materials which are conducive to being used as body supporting devices. Suitable materials include cotton, polyester and vinyl. The sections 1, 2 and 3 are filled with a synthetic or natural substance, such as pea, grain, bean or shell.

The size of each section 1, 2 and 3 corresponds to the size of each spinal region 11, 12 and 13. Thus, the size of each section 1, 2 and 3 will vary according to the length of the user's spine. The cervical section 1 of the therapeutic neutral spine and exercise device is positioned at the level of the cervical spine 11. The thoracic section 2 is positioned at the level of the thoracic spine 12 and the lumbar section 3 is positioned at the level of the lumbar spine 13.

The length of section 1 corresponds to the length of the user's cervical spine 11 from C1 to C7. The length of section 2 corresponds to the length of the user's thoracic spine 12 from T1 to T12. The length of section 3 corresponds to the length of the user's lumbar spine 13 from L1 to L5.

The width of section 1 corresponds to the width of the user's neck. The width of section 2 is no wider than the distance between the scapula. The width of section 3 corresponds to the width of the user's low back.

The thickness of each section 1, 2 and 3 will vary. The thickness of cervical section 1 will be sufficient to facilitate neutral position of the cervical spine 11. The thickness of thoracic section 2 will be sufficient to encourage neutral spinal position of the thoracic spine 12 and allow the shoulders to posteriorly translate toward the rigid surface 15. The thickness of lumbar section 3 will be sufficient to facilitate neutral position of the lumbar spine 13.

The width, thickness and length of each section 1, 2 and 3 ensure that the entire spine 11, 12 and 13 is properly pushed into neutral spinal alignment.

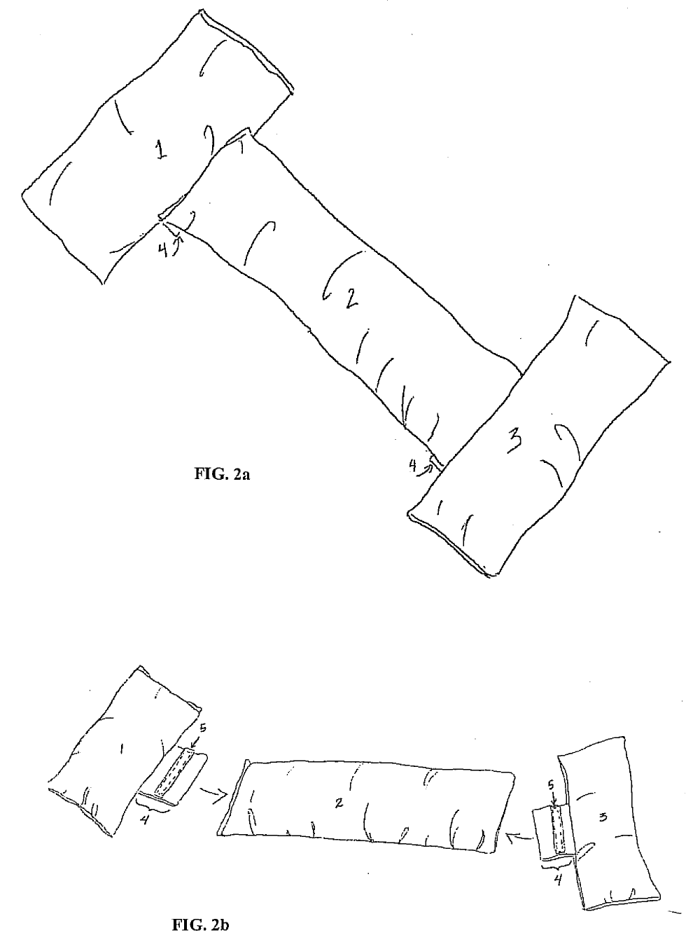

FIG. 2a shows a front view of the therapeutic neutral spine and exercise device as one integral unit in which all three (cervical, thoracic and lumbar) sections are continuous in a horizontal “I” shape.

A preferred embodiment of the present invention is shown in FIG. 2b, in which the front view of each section 1, 2 and 3 detached from one another is depicted. Each section can be used independently by detaching the fastening means 5. Any conventional fastening means such as Velcro®, clasps, buttons or hooks can be used. The fastening means are placed on the tabs 4 of sections 1 and 3. The sections 1 and 2, and 2 and 3 of choice can be attached by fastening means 5 and used together. All sections 1, 2 and 3 can be attached by fastening means 5 and the device can be used as an integral unit.

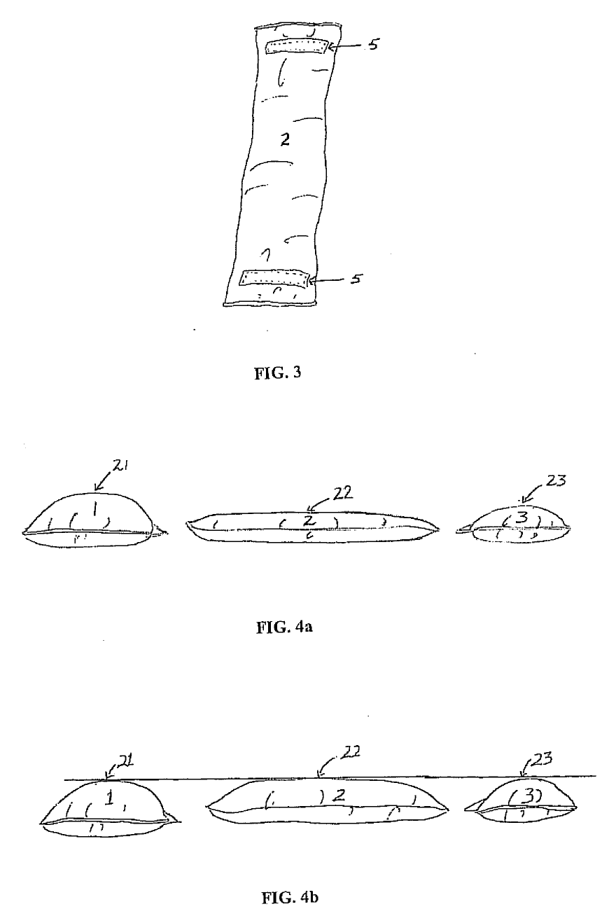

FIG. 3 shows a posterior view of section 2 of the device with fastening means 5.

FIG. 4a shows a side view of sections 1, 2 and 3 of the preferred embodiment of the present invention in which the upper surfaces 21 and 23 of sections 1 and 3 have a typical pillow profile to accommodate, facilitate and encourage neutral position of the cervical spine 11 and lumbar spine 13. Side view of section 2 shows a long and planar or linear shaped profile to its upper surface 22.

FIG. 4b shows a side view of another preferred embodiment of the present invention in which all upper surfaces 21, 22 and 23 of the corresponding cervical 1, thoracic 2 and lumbar 3 sections form a linear profile. This linear profile encourages the reversal of flexed posture assumed throughout the day thus facilitating neutral spinal position.

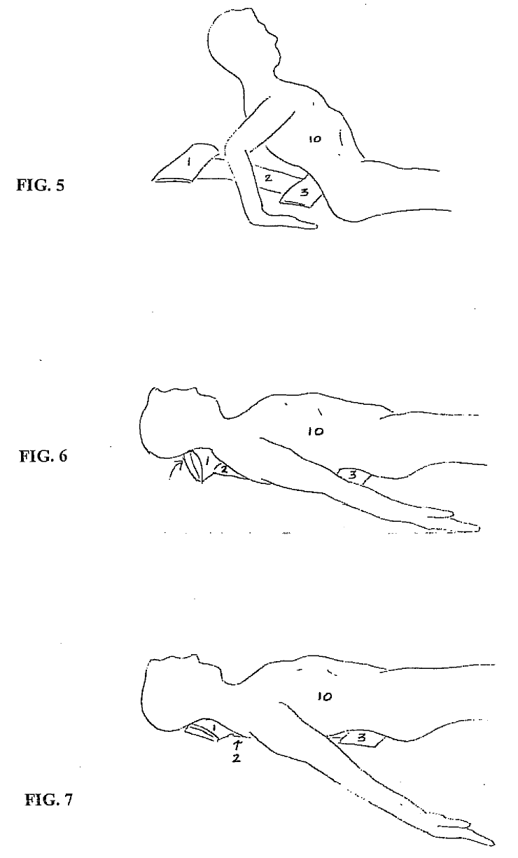

Referring to FIG. 5, to properly use the therapeutic neutral spine and exercise device that is subject of the present invention, the user 10 positions himself on the device. Once positioned, the user lies supine on the sections 1, 2 and 3 that are placed against a rigid surface 15 with cervical section 1 positioned across the cervical spine 11, thoracic section 2 along the center of the entire thoracic spine 12 and lumbar section 3 positioned across the lumbar spine 13 as shown in FIGS. 6 to 9. The weight of the user's body on the sections 1, 2 and 3 fosters and facilitates neutral position of the entire spine 11, 12 and 13 and allows the user's shoulder to translate posteriorly. Thus, the therapeutic neutral spine and exercise device of the present invention enable the user's spine to be pushed and passively stretched into neutral position. This is important because neutral spinal position with a mild posterior shift of the user's shoulders relieves the tension on certain muscle groups and ligaments, facilitates pectoralis and nerve tissue stretch and encourages expansion of the chest wall, which in turn facilitates a proper breathing pattern and distension of the abdominal wall to help stretch any post-surgical abdominal incision.

The sections 1, 2 and 3 can be used in a horizontal, vertical and reclined positions as long as there is a somewhat rigid surface along the posterior aspect of the sections 1, 2 and 3.

FIG. 6 depicts the user 10 properly positioned on the sections 1, 2 and 3 of the present invention; with cervical section 1 placed across the cervical spine 11, thoracic section 2 placed in the center of the user's back along the thoracic spine 12 and lumbar section 3 placed across the lumbar spine 13. FIG. 6 also shows how cervical section 1 can be elevated or adjusted to further facilitate neutral position of the cervical spine 11 which could be straightened secondary to trauma.

FIG. 7 depicts the user 10 lying supine on the sections 1, 2 and 3 and performing a dural/nerve tissue stretch.

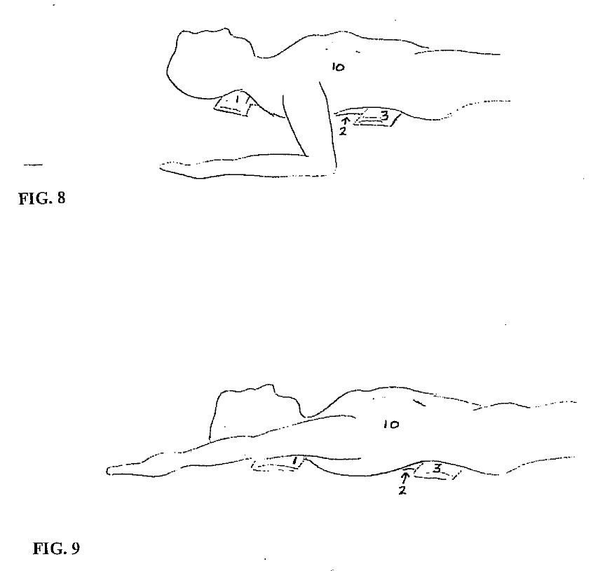

FIG. 8 depicts the user 10 lying supine on the sections 1, 2 and 3 and performing a pectoralis stretch.

FIG. 9 depicts the user 10 lying supine on the sections 1, 2 and 3 and performing a latissimus dorsi stretch.

To administer the therapeutic neutral spine and exercise device of the present invention, a health care provider may use all three sections of the device placed underneath the user or, optionally, use any one of the three sections to facilitate treatment of the specific area(s) of the therapist's choice. For example, if the therapist wishes to treat the user's cervical spine, the therapist can use the cervical section 1 and place it underneath the user's cervical spine.

If a therapist wishes to treat the user's shoulders, the therapist can place all three sections 1, 2 and 3, either as an integral unit or as detached sections, along the spine of the user who lays supine and allows the therapist or user to stretch his pectoralis, latissimus and dural/nerve tissue by varying arm position.

If a therapist wishes to facilitating a stretch of an abdominal incision and diaphragmatic release of the user, the therapist can place all three sections 1, 2 and 3, either as an integral unit or as detached sections, along the spine of the user who lays supine and allow the therapist or user to work on those regions.

If a therapist wishes only to stretch the sub-occipital ligament of a user, the therapist can use the cervical section 1 and place it underneath the user's cervical spine.

Likewise, if a therapist wishes only to stretch the user's abdominal incisions and open the diaphragm, the therapist can use the lumbar section 3 and place it underneath the user's lumbar spine.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. An “I” shaped therapeutic neutral spine and exercise device comprising a cervical section, a thoracic section and lumbar section, wherein:
said cervical section of the device being the length of the cervical spine (C1 to C7) of a user and width corresponding to width of the neck of said user and thickness sufficient to facilitate a neutral position of the cervical spine of said user;
said thoracic section of the pillow being the length of the thoracic spine (T1 to T12) of said user and width being no larger than the distance between the scapula of said user and thickness sufficient to facilitate neutral spinal position and allow the shoulders of said user to translate posteriorly; and
said lumbar section of the pillow being the length of the lumbar spine (L1-L5) of said user and width corresponding to the width of the low back of said user and thickness sufficient to facilitate a neutral position of the lumbar spine of said user;
and all three sections form an integral unit.

2. An “I” shaped therapeutic neutral spine and exercise device comprising a cervical section, a thoracic section and lumbar section, wherein:
said cervical section of the device being the length of the cervical spine (C1 to C7) of a user and width corresponding to width of the neck of said user and thickness sufficient to facilitate a neutral position of the cervical spine of said user;
said thoracic section of the pillow being the length of the thoracic spine (T1 to T12) of said user and width being no larger than the distance between the scapula of said user and thickness sufficient to facilitate neutral spinal position and allow the shoulders of said user to translate posteriorly; and
said lumbar section of the pillow being the length of the lumbar spine (L1-L5) of said user and width corresponding to the width of the low back of said user and thickness sufficient to facilitate a neutral position of the lumbar spine of said user;
and said three sections are demountably attached to each other by fastening means.

3. The therapeutic neutral spine and exercise device according to claim 2 where the fastening means between said cervical, thoracic and lumbar sections of said device is an adhesive, mechanical fastener such as snaps, hooks or Velcro®.

4. The therapeutic neutral spine and exercise device according to claim 1 or claim 2 wherein an upper surface of the cervical and lumbar sections which lies adjacent to the cervical and lumbar spine has a side view of oval or rounded profile shaped to accommodate the contour of the cervical and lumbar spine and facilitate neutral spinal position of said user.

5. The therapeutic neutral spine and exercise device according to claim 1 wherein an upper surface of the thoracic section which lies adjacent to the thoracic spine has a planar or linear side view profile.

6. A method for using the therapeutic neutral spine and exercise device as claimed in claim 1 for treating a cervical spine of a user, comprising placing said detachable cervical section of different width, length aid thickness giving access to a therapist to treat said cervical spine.

7. A method for using the therapeutic neutral spine and exercise device as claimed in claim 1 for treating shoulders of a user, comprising placing said three detachable cervical section, thoracic section and lumbar section along the spine of said user lying supine, allowing a therapist or user to stretch said user's pectoralis, latissimus and dural/nerve tissue by varying arm position.

8. A method for using the therapeutic neutral spine and exercise device as claimed in claim 1 for facilitating a stretch of an abdominal incision and diaphragmatic release in a user, comprising placing said three detachable cervical section, thoracic section and lumbar section along the spine of said user lying supine, allowing a therapist to work on diaphragmatic release or user to stretch said abdominal incision.

9. A method for using the therapeutic neutral spine and exercise device as claimed in claim 1 for stretching the sub-occipital ligament of a user, comprising placing said detachable cervical section underneath said user's cervical spine.

10. A method for using the therapeutic neutral spine and exercise device as claimed in claim 1 for stretching abdominal incisions and opening a diaphragm of a user, comprising placing said detachable lumbar section underneath said user's lumbar spine.
